(19) United States
(12) Patent Application Publication
(10) Pub. No.: US 2010/0256177 A1
(43) Pub. Date: Oct. 7, 2010

(54) Flavopereirine derivatives for cancer therapy

(76) Inventor: Venkatachalam SESHAGIRI, West Bengal (IN)

Correspondence Address: IPHORGAN, LTD., 1130 LAKE COOK ROAD, SUITE 240, BUFFALO GROVE, IL, 60089, US

(21) Appl. No.: 11/629,219

(22) PCT Filed: Jun. 11, 2004
(86) PCT No.: PCT/IN04/00167

§ 371 Date: Jan. 25, 2008

(51) Int. Cl. A61K 31/4375; C07D 471/14; A61P 35/00

(52) U.S. Cl. 514/285; 546/70

(57) ABSTRACT

The present invention relates to novel derivatives of flavopereirine and methods for manufacture thereof. The novel derivatives of flavopereirine are useful for inhibition or prevention of the growth or multiplication of cancer cells, and to therapeutic compositions containing such compounds. The invention more specifically relates to the use of derivatives of flavopereirine for the inhibition and/or prevention of cancer of the ovary, breast, prostate, pancreas, oral cavity, lung, colon, and larynx.

### FIELD OF INVENTION

The present invention relates to novel derivatives of flavopereirine and methods for manufacture thereof. In particular, the present invention relates to the use of novel derivatives of flavopereirine for inhibition or prevention of the growth or multiplication of cancer cells, and to therapeutic compositions containing such compounds. The invention more specifically relates to the use of derivatives of flavopereirine for the inhibition and/or prevention of cancer of the ovary, breast, prostate, pancreas, oral cavity, lung, colon, and larynx.

### BACKGROUND OF THE INVENTION

Most aspects of the cancer problem revolve round the behaviour of DNA. DNA modification by ligand binding starts with the concept of intercalation. The most generally accepted criteria for intercalation come from direct techniques that measure some of the fundamental features. One of them is that the plane of the aromatic chromophore of the bound drug must be parallel to that of the base pairs. In order to have better specificity and selectivity of action we have' synthesized new set of compounds viz. indolo[2,3-a]quinolizinium compounds which are better active than the alkaloid flavopereirine which is known to inhibit cancer producing cells.

### SUMMARY OF THE INVENTION

This invention relates to the synthesis of new indolo[2,3-a]quinolizinium derivatives for cancer therapy having a general structural formula of 1

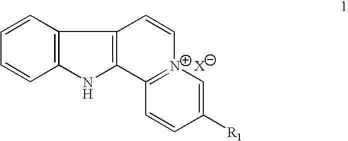

wherein R₁ represent alkyl (not ethyl), alkoxy, aryloxy and their salts thereof; R₁ represent CH(OH)CH₃, CH(OAc)CH₃,CH(OBn)CH₃, OC₂H₅, OBn. where Bn is CH₂C₆H₅.

The present invention also relates to the synthesis of new indolo[2,3-a]quinolizinium derivatives with anticancer activity. The present invention also relates to the design and synthesis of novel flavopereirine derivatives with anticancer activity. The present invention also provides pharmaceutical compositions of novel derivatives of flavopereirine based compounds or pharmaceutically acceptable salts of the flavopereirine based compounds useful for killing or inhibiting multiplication of cancer cells and for testing their bio-activity using cultured human cancer cells as the monitor.

### DETAILED DESCRIPTION OF THE INVENTION

The present invention is directed to novel indolo[2,3-a]quinolizinium derivatives, methods for their manufacture and their use as new cancer agents.

As described herein, the present invention encompasses compounds of the general formula 1 wherein R₁ represent alkyl (not ethyl), alkoxy, aryloxy and their salts thereof.

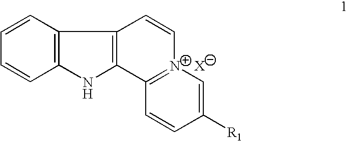

R₁ represent CH(OH)CH₃, CH(OAc)CH₃,CH(OBn)CH₃, OC₂H₅, OBₙ. where Bₙ is CH₂C₆H₅.
Preferred Compounds of Formula 1 are shown below in FIG. 1

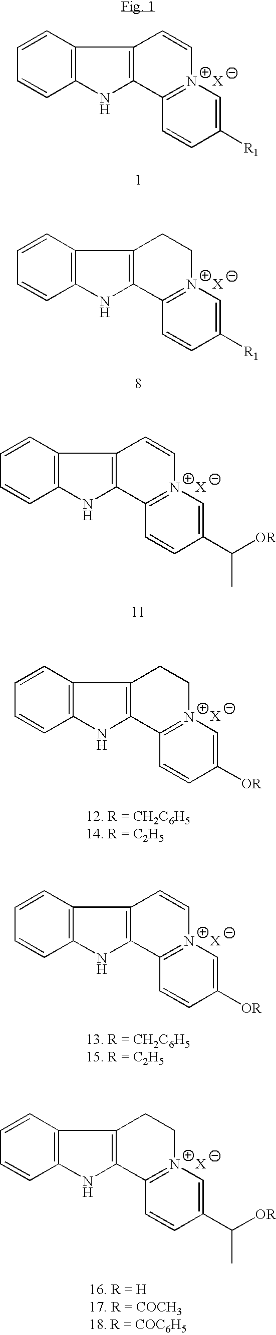

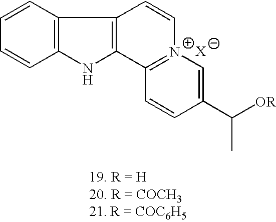

The present invention is also directed to the synthesis of compounds having the structural formula 1.

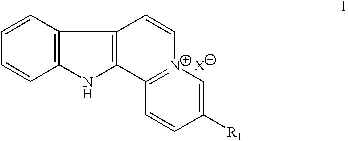

The preferred substitution pattern is aryloxy system in R₁ position.

In another preferred embodiment, the present invention contemplates employing compounds of formula 1 wherein R₁ is an aryloxy group.

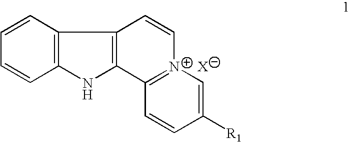

A typical synthesis of indolo[2,3-a]quinolizinium salts of the general formula 1,

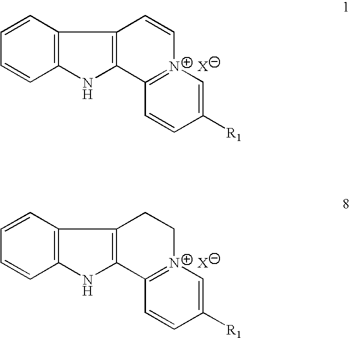

other derivatives of formula 8 and formula 11 are shown in FIGS. 2 and 3, respectively.

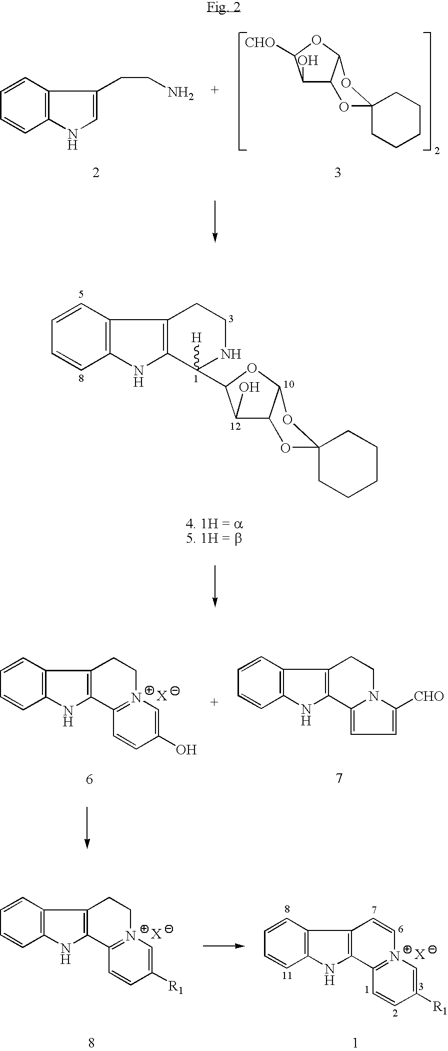

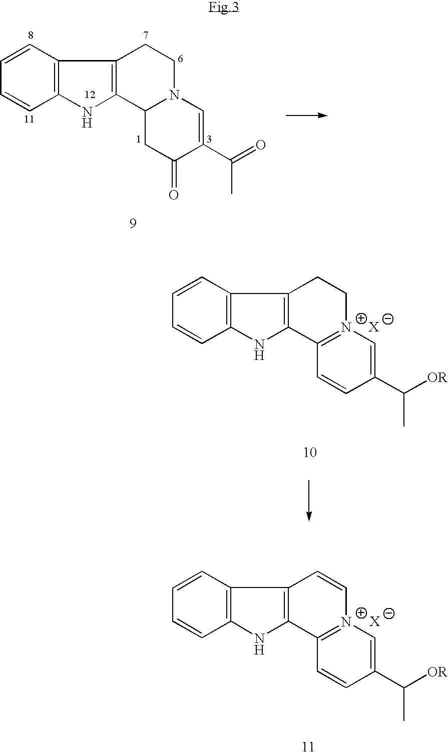

According to one embodiment of the invention, tryptamine of structure 2 is treated with di-(1,2-β-cyclohexylidene-α-D-xylo-pentadialdofuranose-5-hydrate)-5,5′:3′,5-dianhydride of formula 3 in an inert solvent at a temperature in the range of 50° C.-150° C. for a period in the range of 1 to 8 hrs., 1-(10,11-O-cyclohexylidene-12β-hydroxy-13β-tetrahydro-furanyl)tetrahydro-β-carboline of formula 4 and 1-(10,11-O-cyclohexylidene-12β-hydroxy-13α-tetrahydrofuranyl)tetrahydro-β-carboline of formula 5 are isolated and purified.

Thereafter, compounds of formula 4 and 5 are treated with acid at a temperature in the range of −10° C.-100° C. for a period in the range 1 hr. to 8 hrs. and the compounds of formula 6 and formula 7 are recovered by conventional methods.

In an embodiment of the invention inert solvent may be benzene, toluene, xylene.

In an embodiment of the invention acid may be H₂SO₄, HOAc, mixture of H₂SO₄ and HOAc.

Compounds of formula 6 were derivatised to compounds of formula 8 and converted to compounds of formula 1 by known methods. Alternately, they may be oxidized to compounds of formula 1 using different dehydrogenating agents preferably DDQ where DDQ is Dichloro dicyano benzoquinone.

Standard derivatisation techniques have been employed for converting compounds of formula 6.

In the case of compounds of general formula 1 where R₁ is CH(OH)CH₃ or CH(OAc)CH₃ or CH(OBn)CH₃ the derivatives may be prepared from the already known compound, 3-acetyl-1,2,6,7,12,12b-hexahydroindolo[2,3-a]quinolizine-2-one of formula 9. The compound of formula 9 is reduced with NaBH₄ and the resultant mixture of alcohols is isolated by conventional methods. The mixture is refluxed with Hg(OAc)₂ in glacial HOAc to the compound of formula 10 which is derivatised before oxidation with DDQ to formula 11.

Representative salts of the compounds of formula 1 include but are not limited to the following: acetate, ascorbate, benzoate, citrate, oxalate, stearate, trifluoroacetate, succinate, tartarate, lactate, fumarate, gluconate, glutamate, phosphate/diphosphate, and valerate. Other salts include Ca, Li, Mg, Na, and K salts, halides, salts of amino acids such as lysine or arginine; guanidine, ammonium, substituted ammonium salts or aluminium salts. The salts may be prepared in a conventional manner.

The present invention also provides a composition comprising a compound of formula 1, a derivative or salt thereof and a pharmaceutically acceptable carrier, diluent, or solvent. The composition may optionally and preferably contain pharmaceutically acceptable diluents, excipients, additives, fillers, lubricants, solvents, binders, stabilizers, and the like. Such diluents may include: RPMI 1649, buffered saline, isotonic NaCl, Ringer's solution, water, distilled water, polyethylene glycol (neat or in water), 2% Tween in water, dimethyl-sulfoxide to 50% in water, propylene glycol (neat or in water), phosphate buffered saline, balanced salt solution, glycerol, and other conventional fluids that are suitable for intravenous administration. Pharmaceutical compositions, which provide from about 0.1 to 10 gram (preferably 0.5 to 5.0 gram) of the composition per unit dose are preferred and are conventionally prepared as tablets, lozenges, capsules, powders, aqueous or oily suspension, syrups, elixirs, and aqueous solutions. The nature of the pharmaceutical composition employed will, of course, depend on the desired route of administration.

The invention provides a method of treatment for humans, mammals, or other animals suffering from cancer or other tumors. The method may suitably comprise, consist of, or consist essentially of administering a therapeutically effective dose of the pharmaceutical composition so as to kill or inhibit the multiplication of cancer or tumor cells. The invention relates more specifically to the use of the compounds of formula 1 and 8, and their derivatives or salts thereof for the inhibition and/or prevention of cancer of the ovary, breast, prostate, pancreas, oral cavity, lung, colon, and larynx.

The methods of this invention comprise, consist of, or consist essentially of administering systematically to the mammal a therapeutically effective combination of flavoperereine derivatives. An effective dose of flavoperereine derivatives or pharmaceutically acceptable salts of the flavoperereine derivatives ranges from 1 mg/Kg. B. Wt to 300 mg/Kg. B. Wt (preferably 10-100 mg)/Kg. B. Wt) of the mammal, with the dose dependent on the effects sought, the manner of administration, and the cancer being treated. Systemic administration refers to oral, rectal, nasal, transdermal, and parental (i.e., intramuscular, intravenous and subcutaneous). In accordance with good clinical practice, it is preferred to administer the composition at a dose that will produce anticancer effects without causing undue harmful side effects. The composition may be administered either alone or as a mixture with other therapeutic agents such as 5-fluorouracil, methotrexate, etoposide, paclitaxel, taxotere, doxorubicin, daunarubicin, vincristine, vinblastine and other such known and established anticancer drugs.

An effective amount means that amount of a drug or pharmaceutical agent that will elicit the biological or medical response of a tissue, system, animal or human being sought.

Further chemical transformations to obtain the desired molecules were performed using standard methods and some of them have been presented in the following examples.

To further assist in the understanding of the present invention and not by way of limitation the following examples are presented to clearly describe the present invention.

### Example 1

### Preparation of 1-(10,11-O-cyclohexylidene-12β-hydroxy-13β-tetrahydro-furanyl)tetrahydro-β-carboline of formula 4 and 1-(10,11-O-cyclohexylidene-12β-hydroxy-13α-tetrahydrofuranyl)tetrahydro-β-carboline of formula 5

A mixture of 1.6 g (10 mmol) of tryptamine of structure 2 and 2.5 g (5.4 mmol) of the sugar of structure 3 in a 1:1 aqueous methanolic solution (50 ml) of 1 N NaOAc and 1 N HOAc was heated under stirring at 60° C. After most of the starting material has been consumed (TLC monitoring, 5 hrs) the reaction mixture was evaporated under reduced pressure to remove the methanol. It was then neutralized with NaHCO₃ solution and extr-acted with chloroform. The extract was dried (Na₂SO₄), solvent evaporated and the residue chromotographed over basic alumina. Petroleum ether-chloroform (1:1) eluates were combined and concentrated to afford. compound of formula 4 (mp 200-201° C.; 2.04 g; yield, 55%). Chloroform-methanol (9.5:0.5) eluates were combined and concentrated to afford compound of formula 5 (mp 235-237° C.; 1.11 g; yield, 30%)

### Spectral Data of Compound 4:

¹H NMR (CDCl₃): δ 1.4-1.9 (brs, 10H), 2.64-2.84 (m, 2H, H-4), 2.9-3.20 (m, 2H, H-3), 3.3-3.6 (m, 2H, OH, NH), 4.24 (m, 1H, H-11), 4.4-4.60 (m, 3H, H-1, H-12, H-13), 6.04 (d, J=5 Hz, 1H, H-10), 7.04-7.60 (m, 4H, ArH), 8.44 (brs, 1H, NH).

MS m/z (rel. int.): 370 (M⁺, 90), 327 (25), 271 (20), 255 (28), 237 (22), 213 (50), 199 (30), 184 (84), 172 (98), 171 (100).

### Spectral Data of Compound 5:

¹H NMR (CDCl₃): δ 1.3-1.9 (m, 10H), 2.6-2.9 (m, 2H, H-4), 3.01-3.40 (m, 2H, H-3), 4.36-4.39 (m, 2H, OH, NH), 4.45-4.55 (m, 2H), 5.98 (d, J=3.3 Hz, 1H, H-10), 7.04-7.20 (m, 3H, ArH), 7.47 (d, J=7.2 Hz, 1H), 8.20 (brs, NH, 1H).

MS m/z (rel. int.): 370 (M⁺, 95), 327 (20), 271 (20), 255 (30), 237 (22), 21.3 (50), 171 (100).

### Example 2

### Preparation of 3-hydroxy-6,7-dihydroindolo[2,3-a]quinolizinium of formula 6 and 3-formyl-5,6-dihydroindolo[2,3-c]quinolizinium of formula 7

A mixture (1 mmole, 370 mg) of compounds of formula 4 and 5 was stirred in a solution of 0.3 ml conc. H₂SO₄, 1 ml water and 9 ml acetonitrile at room temperature for 72 hrs. The reaction mixture was monitored (TLC) and when no more starting materials remains it was diluted with water, evaporated to dryness and residue chromatographed over silica gel. CHCl₃-petroleum (9:1) eluates afforded the compound of formula 7 (mp 162-164° C.; 24 mg; yield, 10%) Chloroform-methanol (4:1) eluates were combined and concentrated to afford compound of formula 6 isolated as its hydrogensulphate (mp 296-298° C.; 267 mg; yield, 80%).

Spectral data of compound 6:

¹H NMR (DMSO-d₆): δ 3.25 (t, J=7 Hz, 2H), 4.75 (t, J=7 Hz, 2H), 7.10 (t, J=7.5 Hz, 1H), 7.24 (t, J=7.5 Hz, 1H), 7.46 (d, J=8 Hz, 1H), 7.61 (d, J=8 Hz, 2H), 7.92 (d, J=9 Hz, 1H), 8.11 (s, 1H), 11.96 (brs, 1H, NH).

MS m/z: FAB 237 [M⁺]

### Spectral Data of Compound 7:

¹H NMR (CDCl₃): δ 3.4 (t, J=8 Hz, 2H, H-6), 4.8 (t, J=8 Hz, 2H, H-5), 6.38 (d, J=6 Hz, 1H, H-1), 7.02 (d, J=6 Hz, 1H, H-2), 7.08-7.68 (m, 4H, ArH), 8.34 (brs, 1H, NH), 9.58 (s, —CHO).

MS m/z (rel. int.): 236 (M⁺, 100), 235(96), 207 (30), 206 (32).

### Example 3

### Preparation of 3-benzyloxy-6,7-dihydroindolo[2,3-a]quinolizine of formula 12

To a solution of compound of formula 6 (334 mg, 1 mmol) in DMF (1 ml) and triethyl amine (2 ml), benzyl bromide (342 mg, 2 mmol) was added under stirring at room temperature. The reaction mixture was stirred for 8 hrs. The solvents were removed under reduced pressure and the residue was chromatographed over silica gel. Chloroform-methanol (4:1) eluates afforded compound of formula 12 isolated as its perchlorate (mp 278-280° C.; 338 mg; yield, 80%).

Spectral data of compound 12:

¹H NMR (DMSO-d₆): δ 3.36 (t, J=7 Hz, 2H), 4.90 (t, J=7 Hz, 2H), 5.35 (s, 2H), 7.16 (t, J=7.5 Hz, 1H), 7.34 (t, J=7.5 Hz, 1H), 7.41-7.54 (m, 6H), 8.20 (d, J=9 Hz, 1H), 8.40 (d, J=9 Hz, 1H), 8.99 (s, 1H), 12.21 (brs, 1H, NH).

MS m/z: FAB 327 [M⁺]

### Example 4

### Preparation of 3-benzyloxyindolo[2,3-a]quinolizinium of formula 13

To a solution of 100 mg (0.23 mmol) of the perchlorate salt of the compound of formula 12 in glacial HOAc (10 ml) was added 150 mg (0.6 mmol) of DDQ at a time and the reaction mixture heated over steam bath for 6 hrs. The reaction mixture on cooling afforded crystals of the perchlorate of compound of formula 13 (296-298° C.; 68 mg; yield 70%).

### Spectral Data of Compound 13:

¹H NMR (DMSO-d₆): δ 5.39 (s, 2H), 7.36-7.55 (m, 5H), 7.58 (d, J=7.5 Hz, 1H), 7.72 (t, J=7.5 Hz, 1H), 7.85 (d, J=7.5 Hz, 1H), 8.35 (d, J=7.5 HZ, 1H), 8.43 (d, J=7.5 Hz, 1H), 8.83 (d, J=7.5 Hz, 1H), 8.90 (d, J=9 Hz, 1H), 9.01 (d, J=9 Hz, 1H), 9.38 (s, 11-1), 13.36 (brs, 1H, NH).

MS m/z: FAB 325 [M⁺]

### Example 5

### Preparation of 3-ethoxy-6,7-dihydroindolo[2,3-a]quinolizinium of formula 14

To a solution of compound of formula 6 (334 mg, 1 mmol) in DMF (1 ml) and triethyl amine, (2 ml), ethyl bromide (218 mg, 2 mmol) was added under stirring at room temperature. The reaction mixture was stirred for 8 hrs. The solvents were removed under reduced pressure and the residue was chromatographed over silica gel. Chloroform-methanol (4:1) eluates afforded the compound of formula 14 isolated as its perchlorate (mp 305° C. (dec.); 291 mg; yield, 80%).

Spectral Data of Compound 14:

¹H NMR (DMSO-d₆): δ 1.42 (t, J=6.9 Hz, 3H), 4.28 (q, J=6.9 Hz, 2H), 3.34 (t, J=6.9 Hz, 2H), 4.88 (t, J=6.9 Hz, 2H), 7.16 (t, J=7.1 Hz, 1H), 7.33 (t, J=7.1 Hz, 1H), 7.52 (d, J=8.1 Hz, 1H), 7.70 (d, J=8.11-1z, 1H), 8.16 (d, J=9 Hz, 1H), 8.29 (d, J=9 Hz, 1H), 8.84 (s, 1H), 12.17 (brs, NH, 1H).

MS m/z: FAB 265 [M⁺]

### Example 6

### Preparation of 3-ethoxyindolor-2,3-alquinolizinium of formula 15

To a solution of 100 mg (0.28 mmol) of the compound of formula 14 in glacial HOAc (10 ml) was added 150 mg (0.6 mmol) of DDQ at a time and the reaction mixture heated over steam bath for 6 hrs. The reaction mixture on cooling afforded crystals of the perchlorate of compound of formula 15 (310° C. (dec.); 75 mg; yield, 75%).

### Spectral Data of Compound 15:

¹H NMR (DMSO-d₆): δ 1.48 (t, J=6.9 Hz, 3H), 4.32 (q, J=6.9 Hz, 2H), 7.46 (t, J=7.5 Hz, 1H), 7.71 (t, J=7.5 Hz, 1H), 7.83 (d, J=8.1 Hz, 1H), 8.23 (dd, J=1.5 Hz, 9.0 Hz, 1H), 8.40 (d, J=7.8 Hz, 1H), 8.78 (d, J=6.9 Hz, 1H), 8.82 (d, J=9.0 Hz, 1H), 8.95 (d, J=6.9 Hz, 1H), 9.19 (d, J=1.5 Hz, 1H), 13.31 (brs, NH, 1H).

MS m/z: FAB 263 [M⁺]

### Example 7

### Preparation of 3-(α-hydroxyethyl)-6,7-dihydroindolo[2,3-a]quinolizinium of formula 16

The known compound of formula 9 (2.80 gm, 10 mmol) obtained from 1-methyl-3,4-dihydro-β-carboline and hydroxymethylene acetylacetone was taken in methanol (40 ml) and NaBH₄ (1.90 gm, 50 mmol) was added in portions at room temperature. After all the starting material had been reduced (TLC monitoring), water was added to the reaction mixture and it was extracted with chloroform. The extract was dried (Na₂SO₄), evaporated to dryness in vacuum to a solid residue. This residue was dissolved in HOAc (50 ml) and Hg(OAc)₂ (6.37 gm, 20 ml) was added to it. The mixture was refluxed for 5 hrs., Cooled and the solvent removed by rotavapor. The residue was diluted with water and H₂S was passed into it till there was no further separation of HgS. The solution was filtered over a bed of celite and the filtrate evaporated to dryness. The residue was chromatographed over silica gel. Chloroform-MeOH (95:5) eluates afforded the compound of formula 16 isolated as perchlorate salt (mp 300-302° C.; 2.91 g; yield, 80%).

Spectral Data of Compound 16:

¹H NMR (DMSOd₆): δ 1.46 (d, J=7 Hz, 3H), 3.37 (t, J=7 Hz, 2H), 4.94 (t, J=7 Hz, 2H), 4.98 (q, J=7 Hz, 1H), 6.00 (d, J=4 Hz, 1H, OH), 7.16 (t, J=7 Hz, 1H), 7.35 (t, J=7 Hz, 1H), 7.54 (d, J=8 Hz, 1H), 7.71 (d, J=8 Hz, 1H), 8.46 (d, J=8 Hz, 1H), 8.52 (d, J=8 Hz, 1H), 8.95 (s, 1H), 12.92 (brs, 1H, NH).

MS m/z (rel. int.): 265 (vr, 9), 264(47), 263 (100), 247 (19), 219 (12), 218 (7).

### Example 8

### Preparation of 3-(α-acetoxyethyl)-6,7-dihydroindolo[2,3-a]quinolizinium of formula 17

A solution of compound 16 (365 mg, 1 mmol) in pyridine (1 ml) and acetic anhydride (0.5 ml) was heated for 3 hrs over steam bath. The reaction mixture was then concentrated to remove both excess pyridine and acetic anhydride in a rotary evaporator. The residue was chromatographed over silicagel. Chloroform-MeOH (99:1) eluates were combined, concentrated to afford the compound of formula 17 isolated as its perchlorate (mp 298-300° C. (dec.); 386 mg; yield, 95%).

### Spectral Data of Compound 17:

¹H NMR (DMSO-d₆): δ 1.60 (d, J=7 Hz, 3H), 2.14 (s, 3H), 3.40 (t, J=7 Hz, 2H), 4.96 (t, J=7 Hz, 2H), 5.98 (q, J=7 Hz, 1H), 7.08-7.84 (m, 4H), 8.46-8.74 (m, 2H), 9.12 (s, 1H), 12.96 (brs, 1H, NH).

MS m/z: FAB 307 [M⁺]

### Example 9

### Preparation of 3-(α-benzoyloxyethyl)-6,7-dihydroindolo[2,3-a]quinolizinium of formula 18

A solution of compound 16 (365 mg, 1 mmol) in pyridine (1 ml) and benzoyl chloride (0.5 ml) was heated for 3 hrs over steam bath. The reaction mixture was then concentrated to remove both excess pyridine and benzoyl chloride in a rotary evaporator. The residue was chromatographed over silica gel. Chloroform-MeOH (99:1) eluates were combined to afford the compound of formula 18 isolated as chloride salt (mp 288-289° C.; 364 mg; yield, 90%).

Spectral data of compound 18:

¹H NMR (DMSO-d₆): δ 1.72 (d, J=6.5 Hz, 3H), 3.30 (t, J=7.5 Hz, 2H), 4.93 (t, J=7.5 Hz, 2H), 6.21 (q, J=6.5 Hz, 1H), 7.17 (t, J=7.5 Hz, 1H), 7.38 (t, J=7.5 Hz, 1H), 7.5-7.75 (m, 5H), 8.05-8.09 (m, 2H), 8.23 (d, J=7.5 Hz, 1H), 8.73 (d, J=8 Hz, 1H), 9.15 (s, 1H), 12.33 (brs, 1H).

MS m/z: FAB 369 [M⁺]

### Example 10

### Preparation of 3-(α-hydroxyethyl)indolo[2,3-a]quinolizinium of formula 19

To a solution of 100 mg (0.28 mmol) of the compound of formula 16 in glacial HOAc (10 ml) was added 150 mg (0.6 mmol) of DDQ at a time and the reaction mixture heated over steam bath for 6 hrs. Addition of few drops of HClO₄ to the reaction mixture afforded compound of formula 19 as perchlorate salt (mp 288-290° C.; 83 mg; yield, 82%)

### Spectral Data of Compound 19:

¹H NMR (DMSO-d₆): δ 1.53 (d, J=6.5 Hz, 3H), 5.05-5.10 (m, 1H), 5.96 (d, J=4.5 Hz, 1H), 7.49 (t, J=7 Hz, 1H), 7.75 (t, J=7 Hz, 1H), 7.88 (d, J=8.4 Hz, 1H), 8.46 (d, J=7.8 Hz, 1H), 8.84 (d, J=7 Hz, 1H), 8.9 (d, J=9 Hz, 1H), 9.15 (d, J=7 Hz, 1H), 9.4 (brs, 1H).

MS m/z: FAB 263 [M⁺]

### Example 11

### Preparation of 3-(α-acetoxyethypindolo[2,3-a]quinolizinium of formula 20

To solution of 100 mg (0.24 mmol) of the compound of formula 17 in glacial HOAc (10 ml) was added 150 mg (0.6 mmol) of DDQ at a time and the reaction mixture heated over steam bath for 6 hrs. Addition of few drops of HClO₄ to the reaction mixture afforded compound of formula 20 as perchlorate salt (mp 270-272° C.; 68 mg; yield, 70%).

### Spectral Data of Compound 20:

¹H NMR (DMSO-d₆): δ 1.66 (d, J=6.5 Hz, 3H), 2.16 (s, 3H), 6.1 (q, J=6.5 Hz, 1H), 7.5 (t, J=7.5 Hz, 1H), 7.76 (t, J=7.5 Hz, 1H), 7.88 (d, J=8 Hz, 1H), 8.46 (d, J=7.8 Hz, 1H), 8.53 (d, J=9 Hz, 1H), 8.88 (d, J=7 Hz, 1H), 8.95 (d, J=9 Hz, 1H), 9.12 (d, J=7 Hz, 1H), 9.5 (brs, 1H).

MS m/z: FAB 305 [M⁺]

### Example 12

### Preparation of 3-(α-benzoyloxyethyl)indolo[2,3-a]quinolizinium of formula 21

To solution of 100 mg (0.21 mmol) of the compound of formula 18 in glacial HOAc (10 ml) was added 150 mg (0.6 mmol) of DDQ at a time and the reaction mixture was heated over steam bath for 6 hrs. Addition of few drops of HClO₄ to the reaction mixture afforded compound of formula 21 as perchlorate salt (mp 298-300° C.; 72 mg; yield, 74%).

### Spectral Data of Compound 21:

¹H NMR (DMSO-d₆): δ 1.81 (d, J=6.5 Hz, 3H), 6.37 (q, J=6.5 Hz, 1H), 7.48-7.78 (m, 5H), 7.88 (d, J=8.1 Hz, 1H), 8.11 (d, J=7.2 Hz, 2H), 8.47 (d, J=7.8 Hz, 1H), 8.65 (d, J=8.4 Hz, 1H), 8.88 (d, J=6.9 Hz, 1H), 9.05 (d, J=8.4 Hz, 1H), 9.17 (d, J=6.9 Hz, 1H), 9.63 (s, 1H), 13.6 (brs, 1H, NH).

MS m/z: FAB 367 [M⁺]

### Example 13

### In Vitro Cytotoxicity of the Flavopereirine derivatives

A number of the Flavopereirine derivatives were tested for cytotoxicity against nine (9) human tumor cell lines. Briefly, a three day MTT cytotoxicity assay was performed, which is based on the principle of uptake of MTT (3-(4,5-dimethylthiazol-2-yl)-2,5-diphenyl tetrazolium bromide), a tetrazolium salt, by the metabolically active cells where it is metabolized by active mitochondria into a blue colored formazan product that is read spectrophotometrically. MIT was dissolved in phosphate buffered saline with a pH of 7.4 to obtain an MTT concentration of 5 mg/ml; the resulting mixture was filtered through a 0.22-micron filter to sterilize and remove a small amount of insoluble residue. For each type of tumor cell, 10000 to 15000 cells were seeded in a 96-well culture plate and incubated with the individual Flavopereirine derivatives in concentration ranging from 1-20 μg/ml in a CO₂ incubator for a total of 72 hours. Control cells not treated with the Flavopereirine derivatives were similarly incubated. The assay was terminated by adding 100 μg (20 μl) of MTT to each well, then incubating for additional one hour, and finally adding 50 μl of 10% SDS-0.01N HCl to each well to lyse the cells and dissolve formazan. After incubating for one hour, the plate was read spectrophotometrically at 540 nm and the percentage of cytotoxicity calculated using the following formula:

Cytotoxicity percentage=100×[1-(X/R₁)], where X=(absorbance of treated sample at 540 nm)−(absorbance of blank at 540 nm) R₁=absorbance of control sample at 540 nm.

The IC₅₀ Values of the cytotoxicity defined as the concentration at which 50% of the cells are killed in vitro was calculated for each cell line treated with each of the Flavopereirine derivatives.

The cell lines are ovary (PA-1), breast (MCF-7), prostate (DU145), pancreas (MiaPaCa.2), oral cavity (KB), lung (L132), colon (SW620), and larynx (HeP2). The flavopereirine derivatives tested for their cytotoxicity are shown in table 1. The ED50 values of in vitro cytotoxicity of the flavoperereine derivatives of this invention are shown in the Table 2.

TABLE 1
Flavopereirine derivatives (22-33)

22-27

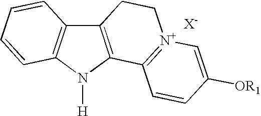

28-33

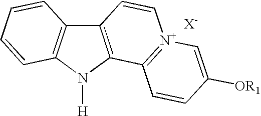

| Compound | R₁ | X | Compound | R₁ | X |
|---|---|---|---|---|---|
| 22 | H | HSO₄ | 28 | CH₂CH₆H₅ | ClO₄ |
| 23 | CH₂CH₆H₅ | ClO₄ | 29 | CH₂CH₃ | ClO₄ |
| 24 | CH₂CH₃ | ClO₄ | 30 | (CH₂)₃Br | ClO₄ |
| 25 | CH₂CH₆H₄F[4-] | ClO₄ | 31 | CH₂CH₆H₅ | Br |
| 26 | CH₂CH₆H₄Br[4-] | Br | 32 | CH₂CH₆H₄F[4-] | ClO₄ |
| 27 | (CH₂)₃CH₃ | Br | 33 | (CH₂)₃CH₃ | Br |

TABLE 2
IC₅₀ (μg/mL) of Flavoperereine derivatives in human tumor cell lines

| | Cell line | | | | | | | | |
|---|---|---|---|---|---|---|---|---|---|
| | PA1 | MCF7 | DU145 | MiaPaca2 | KB | L132 | Hep2 | SW620 | ECV-304 |
| Compound | Ovarian | Breast | Prostate | Pancreas | Oral | Lung | Larynx | Colon | Endothelial |
| 22 | >100 | >100 | >100 | >100 | >100 | >100 | >100 | >100 | ND |
| 23 | 31 | 5 | 10 | 10 | 12 | 20 | 17 | 11 | 16 |
| 24 | 38 | ND | ND | ND | 50 | 20 | 89 | 30 | 64 |
| 25 | 36 | 34 | 41 | 30 | 8 | 9 | 44 | 44 | 24 |
| 26 | >100 | >100 | 7.5 | 9.5 | 6 | ND | 24 | >100 | 12 |
| 27 | 4.7 | 6 | 9 | 7.5 | 7 | ND | 5 | 4 | ND |
| 28 | 1.5 | 5 | 2 | 2 | 2 | 2 | 1.5 | <1 | 4 |
| 29 | 5 | ND | ND | ND | 10 | 15 | 8.0 | 10 | 4 |
| 30 | 2 | 6 | 7.5 | 4 | 10 | 10 | 1.5 | 6 | 6 |
| 31 | 4 | 4 | 4.5 | <1 | <1 | 4.5 | 3 | 5 | 13.5 |
| 32 | 6 | <1 | 2 | <1 | <1 | <1 | 4 | 5 | 24 |
| 33 | <1 | 3 | 5 | 2 | 1.6 | ND | 2.4 | 1.4 | ND |

A Pub Med search of the National Library of Medicine was carried out to determine the relevance of cell lines used by us for determining the anticancer activity of the peptides. While DU145 (human prostate) showed 542 “hits” when searched with reference to cancer, other human cancer cell lines used by us also showed large number of hits (1063 for MCF-7 and 182 for PA-1). This clearly shows the extensive use of these cell lines in cancer research. Further, it is a common and standard practice and norm for testing molecules for anticancer activity in vitro on human tumor cell lines. (Br J. Cancer. 2001 May 18; 84(10): 1289-90 (Flasks, Fibres & Flanks-Preclinical tumor models for predicting clinical antitumor activity). The authors report that in vitro activity against 6 or more lung or breast cancer cell lines does predict xenograft activity against these tumor types. In articles “Semin Oncol 1992 December; 19(6): 622-38 (The National Cancer Institute: cancer drug discovery and development program) and “Jpn J Antibiot 1977 December; 30 Supp1:35-40 (Antitumor screening procedures of the National Cancer Institute)” extensive use of human tumor cell lines for identification of potential cytotoxic drugs is described.

We claim:

1. A compound of formula 1 indolo[2,3-a]quinolizinium salts of the general formula 1 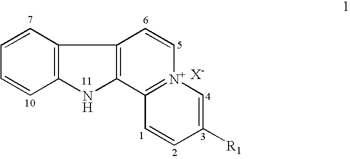
derivatives or salts thereof, wherein R₁ represent alkyl (not ethyl), alkoxy, aryloxy and their salts thereof; CH(OH)CH₃, CH(OAc)CH₃,CH(OBn)CH₃, OC₂H₅, OBn. where Bn is CH₂C₆H₅.

2. A compound of the formula 8, derivatives or salts thereof, wherein R₁ represent alkyl (not ethyl), alkoxy, aryloxy and their salts thereof; R₁ represent CH(OH)CH₃, CH(OAc)CH₃,CH(OBn)CH₃, OC₂H₅, OBn. where Bn is CH₂C₆H₅.

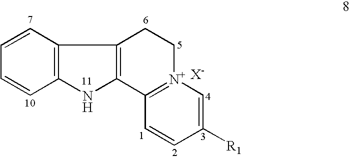

3. A compound according to claim 1, wherein the indolo[2,3-a]quinolizinium is a tetracyclic indolo[2,3-a]quinolizinium substituted at the 3 position shown as R₁ in the formula 1 and their salts thereof.

4. A compound according to claim 2, wherein the indolo[2,3-a]quinolizinium is a tetracyclic 5,6-dihydro indolo[2,3-c]quinolizinium substituted at the 3 position shown as R₁ in the formula 8 and their salts thereof.

5. A compound according to claim 1, having formula 13 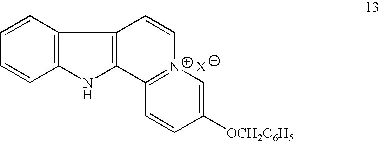
and is 3-benzyloxyindolo[2,3-a]quinolizinium salt.

6. A compound according to claim 1, having formula 15 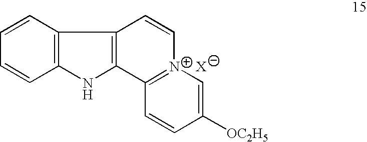
and is 3-ethoxyindolo[2,3-a]quinolizinium salt.

7. A compound according to claim 1, having formula 19 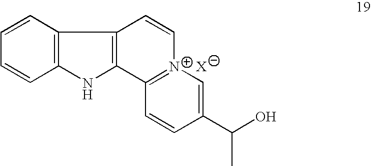
and is 3-(α-hydroxyethypindolo[2,3-a]quinolizinium salt.

8. A compound according to claim 1, having formula 20 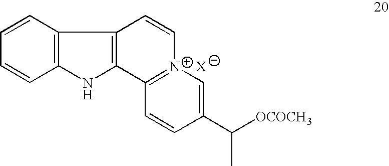
and is 3-(α-acetoxyethyl)indolo[2,3-a]quinolizinium salt.

9. A compound according to claim 1, having formula 21 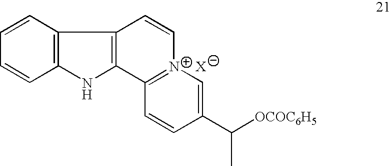
and is 3-(α-benzoyloxyethyl)indolo[2,3-a]quinolizinium salt.

10. A compound according to claim 2, having formula 12 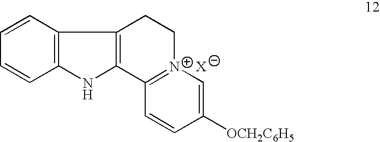
and is 3-benzyloxy-5,6-dihydroindolo[2,3-a]quinolizinium salt.

11. A compound according to claim 2, having formula 14 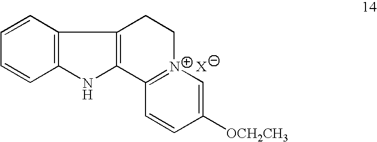
and is 3-ethoxy-5,6-dihydroindolo[2,3-a]quinolizinium salt

12. A compound according to claim 2, having formula 16 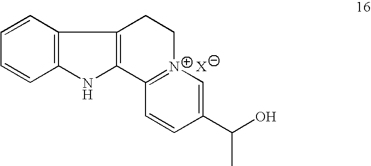
and is 3-(α-hydroxyethyl)-5,6-dihydroindolo[2,3-a]quinolizinium salt

13. A compound according to claim 2, having formula 17 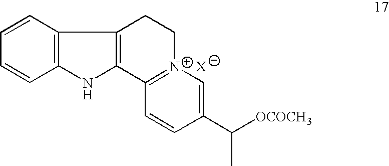
and is 3-(α-acetoxyethyl)-5,6-dihydroindolo[2,3-a]quinolizinium salt

14. A compound according to claim 2, having formula 18 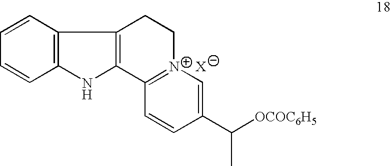
and is 3-(α-benzoyloxyethyl)-5,6-dihydroindolo[2,3-a]quinolizinium salt

15. A composition comprising a compound of any one of claims 1-14, and a pharmaceutically acceptable additive, diluent, excipient, solvent, binder, stabilizer, carrier, filler or lubricant.

16. A composition as claimed in claims 1, 5-9 and 15, which provide 0.1 to 10 gram per unit dose of the compound of formula 1, a salt or derivative thereof.

17. A composition as claimed in claims 2, 10-14 and 15, which provide 0.1 to 10 gram per unit dose of the compound of formula 8, a salt or derivative thereof.

18. The composition as claimed in claims 1-17 in the form of a tablet, lozenge, capsule, powder, aqueous or oily suspension, syrup, elixir, implant or aqueous solution.

19. A method for treating a patient with cancer of the ovary, breast, prostate, pancreas, oral cavity, lung, colon, and larynx comprising administering an effective amount of a compound of formula 1 or 8 as claimed in any one of the claims 1-14 or a composition as claimed in any one of claims 15-18 to the patient in need thereof.

20. A method as claimed in claim 19, wherein said patient is a human, mammal or other animal.

21. The method as claimed in claims 19 and 20 wherein the dosage for humans is in the range of 1 mg/Kg. B. Wt to 300 mg/Kg. B. Wt.

22. The method as claimed in claims 19 and 20 wherein the dosage for humans is in the range of 1 mg/Kg. B. Wt to 300 mg/Kg. B. Wt.

23. The method as claimed in claims 19-21, wherein the compound is administered to the patient systemically

24. Use of the compound of Formula 1 as claimed 1 in preparation of medicines for the treatment of cancer of the ovary, breast, prostate, pancreas, oral cavity, lung, colon, and larynx.

25. Use of the compound of Formula 8 as claimed 2 in preparation of medicines for the treatment of cancer of the ovary, breast, prostate, pancreas, oral cavity, lung, colon, and larynx.

26. Method for the synthesis of indolo[2,3-a]quinolizinium salts of the general formula 1 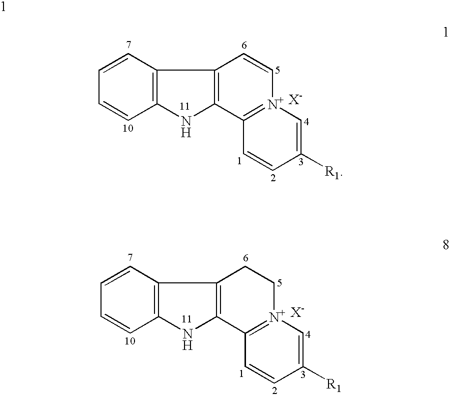
wherein R₁ represent alkyl (not ethyl), alkoxy, aryloxy and their salts thereof R₁ represent CH(OH)CH₃, CH(OAc)CH₃,CH(OBn)CH₃, OC₂H₅, OBn. where Bn is CH₂C₆H₅, which comprises treating a tryptamine of formula 2 with di-(1,2-O-cyclohexylidene-α-D-xylo-pentadialdofuranose-5-hydrate)-5,5′;3′,5-dianhydride of formula 3 in an inert solvent at a temperature in the range of 50° C.-150° C. for a period in the range of 1 to 8 hrs., isolating and purifying 1-(10,11-O-cyclohexylidene-12β-hydroxy-13β-tetrahydro-furanyl) tetrahydro-β-carboline of formula 4 and 1-(10,11-O-cyclohexylidene-12β-hydroxy-13α-tetrahydrofuranyl)tetrahydro-β-carboline of formula 5 so produced, treating said compounds of formula 6 and formula 7, derivatising said compounds of formula 6 to compounds of formula 8 and converted said compounds of formula 8 to compounds formula 1 by known methods.

27. A process as claimed in claim 25 wherein said compound of formula 6 is oxidized to compounds of formula 9 in the presence of a dehydrogenating agent, preferably DDQ where DDQ is Dichloro dicyano benzoquinone

28. A process as claimed in claim 25 or 26 wherein inert solvent is selected from benzene, toluene, xylene.

29. A process as claimed in any one of the claims 25 to 28 said acid is H₂SO₄, HOAc, or a mixture thereof.

30. A process as claimed in claim 26 wherein said dehydrogenating agent is Dichloro dicyano benzoquinone.
